(19) United States
(12) Patent Application Publication
(10) Pub. No.: US 2017/0071741 A1
(43) Pub. Date: Mar. 16, 2017

(54) OSTEOSYNTHETIC IMPLANT

(71) Applicant: OLYMPUS CORPORATION, Tokyo (JP)

(72) Inventor: Hirofumi TANIGUCHI, Tokyo (JP)

(73) Assignee: OLYMPUS CORPORATION, Tokyo (JP)

(21) Appl. No.: 15/358,456

(22) Filed: Nov. 22, 2016

Related U.S. Application Data
(63) Continuation of application No. PCT/JP2015/056166, filed on Mar. 3, 2015.

(30) Foreign Application Priority Data
Jun. 3, 2014 (JP) 2014-114978

(51) Int. Cl. A61F 2/28

(52) U.S. Cl. CPC A61F 2/28; A61F 2310/00041; A61F 2310/00592; A61F 2310/00598

(57) ABSTRACT

Provided is an osteosynthetic implant including a base material composed of magnesium or a magnesium alloy and a ceramic membrane containing magnesium and formed on a surface of the base material. The ceramic membrane includes a porous lower membrane layer disposed in a region adjacent to the base material and an upper membrane layer covering the lower membrane layer and serving as an outermost layer. The upper membrane layer is denser than the lower membrane layer.

### CROSS-REFERENCE TO RELATED APPLICATIONS

This is a continuation of International Application PCT/JP2015/056166 which is hereby incorporated by reference herein in its entirety.

This application is based on Japanese Patent Application No. 2014-114978, the contents of which are incorporated herein by reference.

### TECHNICAL FIELD

The present invention relates to osteosynthetic implants.

### BACKGROUND ART

A known biodegradable implant material in the related art achieves high corrosion resistance within a biological organism by having a porous membrane formed on a base material composed of a magnesium alloy (for example, see Patent Literature 1).

### CITATION LIST

### Patent Literature

### {PTL 1}

PCT International Publication No. WO 2013/070669

### SUMMARY OF INVENTION

### Solution to Problem

An aspect of the present invention provides an osteosynthetic implant including a base material composed of magnesium or a magnesium alloy and a ceramic membrane containing magnesium and formed on a surface of the base material. The ceramic membrane includes a porous lower membrane layer disposed in a region adjacent to the base material and an upper membrane layer covering the lower membrane layer and serving as an outermost layer. The upper membrane layer is denser than the lower membrane layer.

### BRIEF DESCRIPTION OF DRAWINGS

FIG. 1 is a vertical sectional view illustrating a surface region of an osteosynthetic implant according to an embodiment of the present invention.

FIG. 2 illustrates a transmissive-electron-microscope image showing a cross section of a thin film sample fabricated in accordance with the FIB (focused ion beam) technique, the cross section corresponding to an area from a base material to an upper membrane layer of the osteosynthetic implant in FIG. 1.

FIG. 3 illustrates an electron-beam diffraction image of the upper membrane layer of the osteosynthetic implant in FIG. 1.

FIG. 4 illustrates an electron-beam diffraction image of a lower membrane layer of the osteosynthetic implant in FIG. 1.

FIG. 5 illustrates an electron-beam diffraction image of the base material of the osteosynthetic implant in FIG. 1.

FIG. 6 is a spectral diagram illustrating results obtained by performing an elemental analysis on the upper membrane layer of the osteosynthetic implant in FIG. 1 in accordance with the angular resolution technique.

FIG. 7 illustrates a scanning-electron-microscope image of the upper membrane layer of the osteosynthetic implant in FIG. 1.

FIG. 8 is a graph illustrating temporal changes in the amount of elution of magnesium ions when the osteosynthetic implant in FIG. 1 is immersed in a phosphate buffer solution.

### DESCRIPTION OF EMBODIMENT

An osteosynthetic implant according to an embodiment of the present invention will be described below with reference to the drawings.

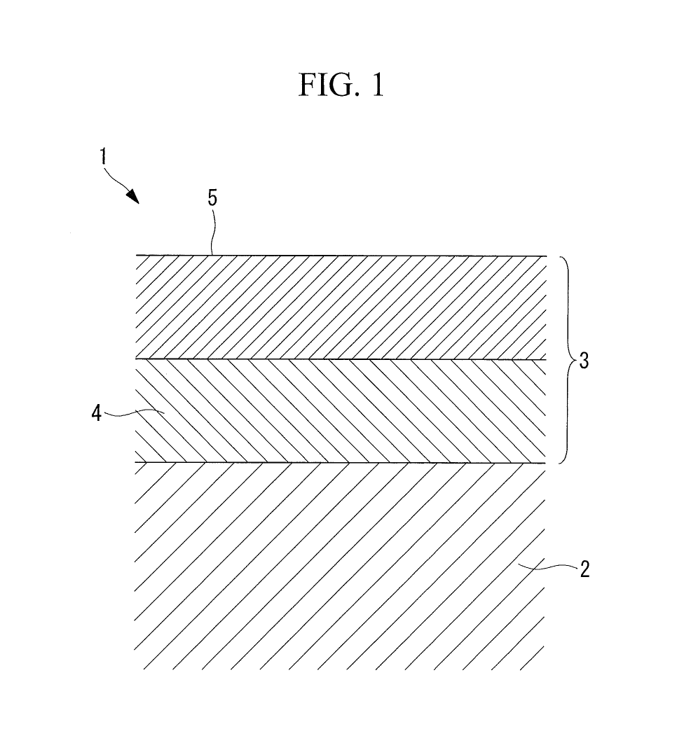

As shown in FIG. 1, an osteosynthetic implant 1 according to this embodiment includes a base material 2 composed of magnesium or a magnesium alloy and a ceramic membrane 3 containing magnesium and formed on the surface of the base material 2.

The ceramic membrane 3 includes a porous lower membrane layer 4 covering the surface of the base material 2 and an upper membrane layer 5 covering the surface of the lower membrane layer 4 and being denser than the lower membrane layer 4. The overall thickness of the ceramic membrane 3 is between 0.1 μm and 12 μm.

The upper membrane layer 5 is amorphous, whereas the lower membrane layer 4 is a mixture of amorphous and crystalline.

The thickness of the upper membrane layer 5 is set such that the period it takes for the upper membrane layer 5 to disappear as a result of being biodegraded by body fluid corresponds to the period it takes for bone fusion to complete, which is, for example, three to twelve weeks. For example, the thickness is set between 0.01 μm and 10 μm.

The lower membrane layer 4 has pores each having a maximum diameter of 200 μm or smaller.

The crystals contained in the lower membrane layer 4 each have a particle diameter smaller than 500 nm.

The effects of the osteosynthetic implant 1 according to this embodiment having the above-described configuration will be described below.

When the osteosynthetic implant 1 according to this embodiment is implanted in bone tissue, the upper membrane layer 5 disposed at the outermost surface comes into contact with the body fluid, so that biodegradation commences.

Since the upper membrane layer 5 is denser than the lower membrane layer 4, the contact area with the body fluid is not very large. In addition, since the upper membrane layer 5 is an amorphous membrane, biodegradation proceeds slowly without intergranular corrosion. Because the thickness of the upper membrane layer 5 is set such that the period it takes for the upper membrane layer 5 to disappear as a result of biodegradation is three to twelve weeks, the osteosynthetic implant 1 can maintain its mechanical strength until the upper membrane layer 5 disappears as a result of biodegradation, so that fusion with the surrounding body tissue can be completed in a stable manner.

When the upper membrane layer 5 disappears as a result of biodegradation, the body fluid reaches the lower membrane layer 4. Since the lower membrane layer 4 is porous, the contact area thereof with the body fluid is larger than that of the upper membrane layer 5, and intergranular corrosion occurs at the crystalline interface contained in the upper membrane layer 5. Thus, the decomposition rate increases. Therefore, the lower membrane layer 4 disappears as a result of biodegradation within a shorter period of time than the time it takes for the upper membrane layer 5 to disappear.

In this case, since the lower membrane layer 4 is also a ceramic layer having amorphous sections, biodegradation proceeds slowly, as compared with the base material 2 composed of magnesium or a magnesium alloy. Thus, a rapid biodegradation process can be suppressed. After the lower membrane layer 4 disappears as a result of biodegradation, the base material 2 comes into contact with the body fluid and quickly disappears as a result of biodegradation. Thus, the osteosynthetic implant 1 can be prevented from remaining as a foreign object inside the body.

Accordingly, the osteosynthetic implant 1 according to this embodiment is advantageous in that it can maintain its mechanical strength from the initial stage of the implanting process to the completion of bone fusion so that the bone fusion can be performed in a stable manner, and that the osteosynthetic implant 1 quickly disappears upon completion of the bone fusion so as to be prevented from becoming a foreign object.

Although the overall thickness of the ceramic membrane 3 is between 0.1 μm and 12 μm and the thickness of the upper membrane layer 5 is between 0.01 μm and 10 μm in this embodiment, the thickness of the lower membrane layer 4 is preferably smaller than 70% of the overall thickness of the ceramic membrane 3. Thus, the time it takes for the body fluid to reach the lower membrane layer 4 can be sufficiently ensured.

Next, a method for manufacturing the osteosynthetic implant 1 according to this embodiment will be described below.

In order to manufacture the osteosynthetic implant 1 according to this embodiment, the base material 2 composed of magnesium or a magnesium alloy is immersed in an electrolytic solution with a pH value ranging between 8 and 13 and containing 0.0001 mol/L to 5 mol/L inclusive of phosphoric acid or a phosphate and 0.01 mol/L to 5 mol/L inclusive of ammonia or ammonium ions but not containing elemental fluorine. Then, an anodizing process is performed on the base material 2 by applying electricity thereto. When applying electricity to the base material 2, the temperature of the electrolytic solution is preferably controlled between 5° C. and 50° C. inclusive.

Before performing the anodizing process, it is preferable that the base material 2 be immersed in acid and alkaline solutions. Thus, for example, a naturally oxidized film on the surface of the magnesium or magnesium alloy and impurities, such as working oil or a release agent used during a shaping process, can be dissolved and removed, thereby improving the quality of an anodized film. Furthermore, immersing the base material 2 in both the acid solution and the alkaline solution is more preferable since insoluble impurities produced when the base material 2 is immersed in one of the solutions can be dissolved and removed by being immersed in the other solution. Examples of the acid solution that can be used include a hydrochloric acid solution, a sulfuric acid solution, and a phosphoric acid solution, and examples of the alkaline solution that can be used include a sodium hydroxide solution and a potassium hydroxide solution. Furthermore, although the solutions used for the immersion process are still effective with their temperatures set at room temperature, performing the immersion process in a state where the solutions are kept between 40° C. and 80° C. is expected to be more effective for dissolving and removing impurities.

The anodizing process is performed by connecting a power source between the base material 2 immersed in an electrolytic solution and serving as an anode and a cathode material similarly immersed in the electrolytic solution.

Although the power source used is not limited in particular and may be a direct-current power source or an alternating-current power source, a direct-current power source is preferred.

In the case where a direct-current power source is used, it is preferable that a constant-current power source be used. The cathode material used is not limited in particular. For example, stainless steel may be suitably used. The surface area of the cathode is preferably larger than the surface area of the base material 2 to be anodized.

The electric current density at the surface of the base material 2 when a constant-current power source is used as the power source is 15 A/dm² or higher. The electricity application time is between 10 seconds and 1000 seconds. When electricity is to be applied by using a constant-current power source, the applied voltage is low at the start of the electricity application process but increases as time elapses. The final applied voltage when the electricity application process ends is 200 V or higher.

Accordingly, with a single anodizing process, an osteosynthetic implant 1 having a ceramic membrane 3 constituted of a lower membrane layer 4 and an upper membrane layer 5 laminated on the surface of the base material 2 can be manufactured.

Next, samples of three types of osteosynthetic implants 1 are fabricated under different processing conditions in accordance with the above-described manufacturing method.

Specifically, as a preliminary process, the base material 2 composed of a magnesium alloy is immersed in 5.7 mol/L phosphoric acid (70° C.), and the surface of the base material 2 is subsequently rinsed with water. Then, the base material 2 is immersed in a 3.8 mol/L sodium hydroxide solution (70° C.), and the surface of the base material 2 is subsequently rinsed with water.

An electrolytic solution containing 0.05 mol/L of a phosphate and 1.9 mol/L of ammonia or ammonium ions is prepared, and the temperature thereof is controlled to 10° C. The water-rinsed base material 2 serving as an anode is immersed in this electrolytic solution and is anodized at an electric current density of 20 A/dm² by using an SUS304 material as a cathode, whereby samples are fabricated. In this case, the voltages reached are 300 V (sample A), 400 V (sample B), and 500 V (sample C).

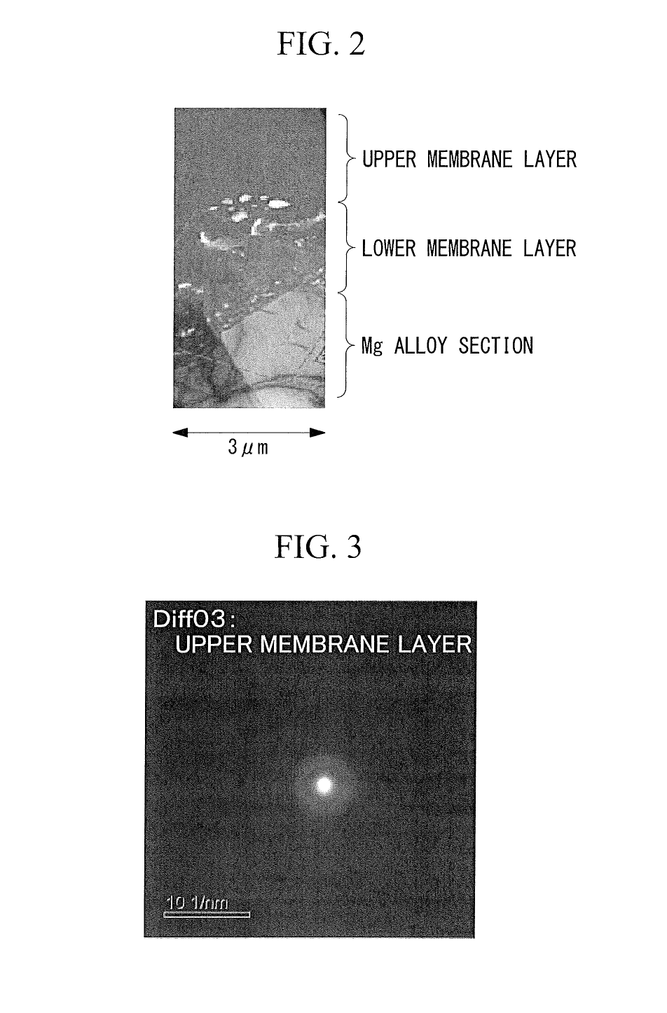

With regard to each of the manufactured samples A, B, and C, a thin film sample is fabricated in accordance with the FIB technique and is observed with an electronic microscope. As a result, as shown in FIG. 2, a lower membrane layer adjacent to the base material 2 and an upper membrane layer adjacent to the lower membrane layer are observed in the vertical section of each of the samples A, B, and C. According to this, it is clear that the lower membrane layer adjacent to the base material 2 has cavities and is thus porous, and that the upper membrane layer has a smaller number of cavities than the lower membrane layer and is thus denser.

Results obtained by checking the average thickness of the ceramic membrane from the electron-microscope observation images indicate 0.8 μm (for sample A), 2.1 μm (for sample B), and 5.3 μm (for sample C). The thickness of the lower membrane layer 4 is 0.3 μm (for sample A), 1.5 μm (for sample B), and 6.1 μm (for sample C).

Furthermore, as a comparative sample, a sample anodized under conditions indicated in Example 6 of PCT International Publication No. WO 2013/070669, which is a superficially-porous sample, is fabricated.

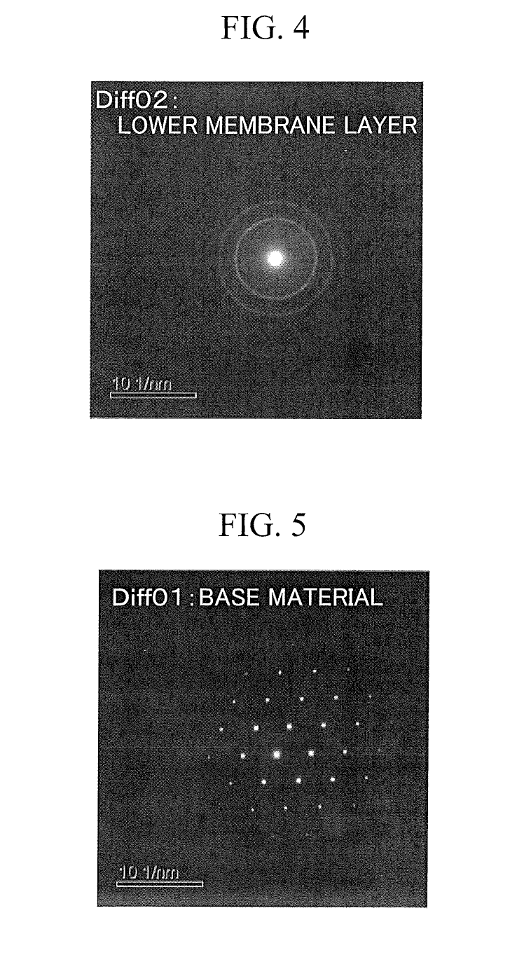

Furthermore, an electron beam of 500 nm diameter is radiated onto the thin film sample of each of the samples, and a diffraction image thereof is observed. As a result, in each of the samples, the electron-beam diffraction image has no rings or spots appearing in the upper membrane layer 5, which indicates that it is amorphous, as shown in FIG. 3. In the lower membrane layer 4, a mixed-crystal structure having both crystals with a size smaller than 500 nm and an amorphous structure is confirmed from diffraction-line rings, as shown in FIG. 4. In the base material 2, a mono-crystal structure with a size of 500 nm or larger is confirmed from diffraction-line spots, as shown in FIG. 5.

Furthermore, as a result of measuring the distances between crystal faces in the electron-beam diffraction diagram of the lower membrane layer 4 shown in FIG. 4, distances of 0.151 nm and 0.215 nm are obtained. These distances between faces are substantially equal to the distance between the faces of magnesium oxide and thus indicate the presence of magnesium oxide crystals.

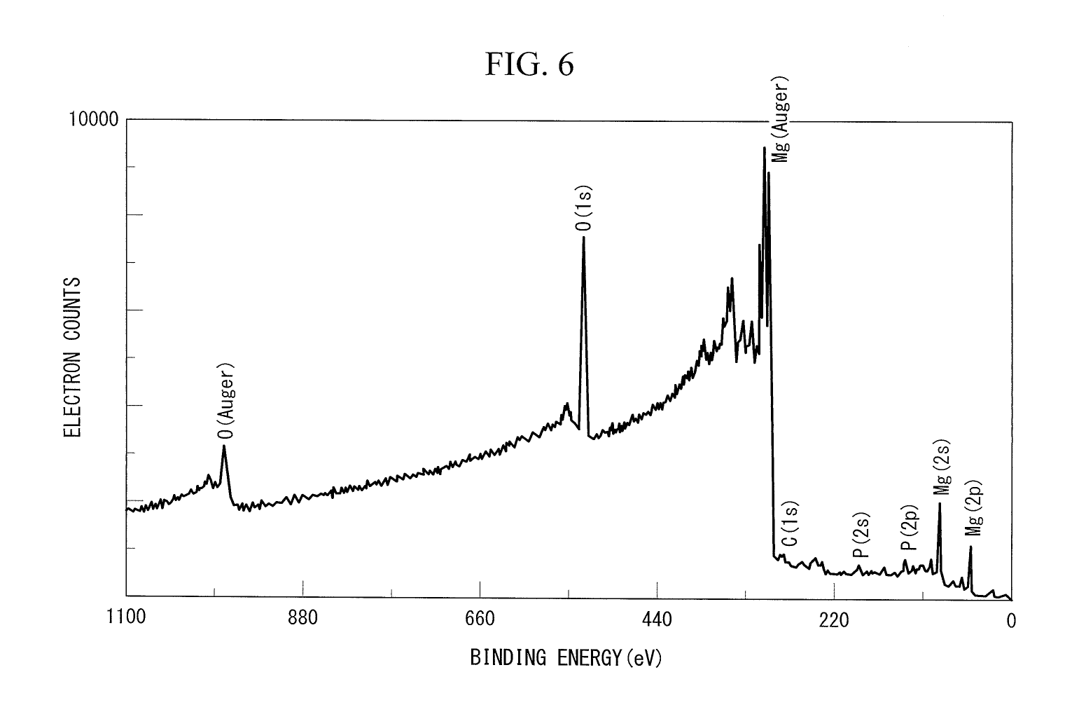

Furthermore, as a result of performing a quantitative elemental analysis on the upper membrane layer 5 in accordance with the angular resolution technique, it is clear that the upper membrane layer contains O, Mg, C, and P elements, as indicated in Table 1 obtained from a wide-scan spectrum shown in FIG. 6. Moreover, since the amount of C tends to decrease in the depth direction of the ceramic membrane 3, it is clear that C is an impurity and that the main components are O, Mg, and P.

TABLE 1

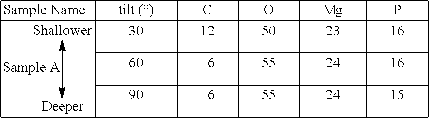

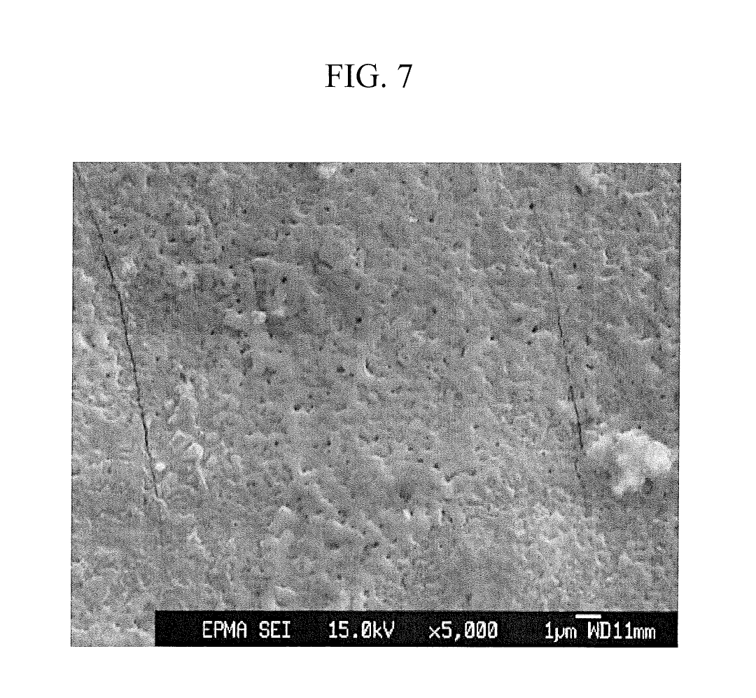

FIG. 7 illustrates a scanning-electron-microscope image of the upper membrane layer 5 of a sample. According to this, pores with a diameter of 0.2 μm or larger are not observed in the surface of the upper membrane layer 5.

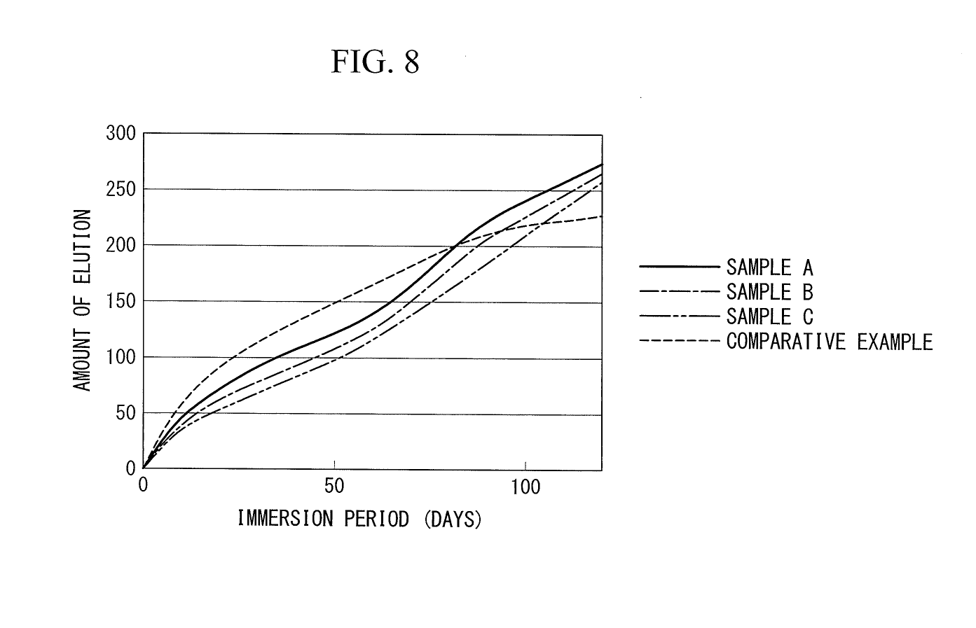

FIG. 8 illustrates temporal changes in the amount of elution of magnesium ions in a case where the three types of samples described above and the sample according to the comparative example are immersed in a phosphate buffer solution. The amount of elution is quantitatively measured in accordance with ICP (inductively coupled plasma-atomic emission spectroscopy).

According to this, the amount of elution of magnesium ions immediately after immersion is kept smaller in the three samples than in the comparative example, meaning that the three samples have higher corrosion resistance than the comparative example. Then, after about 90 days from the immersion, the amount of elution becomes larger than in the comparative example. In other words, the three samples are biodegraded slowly at the initial stage of the implanting process, and then the decomposition rate increases from after about 90 days.

It is said that autologous human bone fusion takes approximately three to twelve weeks. The mechanical strength of the osteosynthetic implant can be maintained by slowing down the progress of biodegradation during the twelve weeks, and the decomposition rate is increased after the twelve weeks so that the osteosynthetic implant can be made to quickly disappear.

Although a magnesium oxide membrane formed by an anodizing process is described as an example of the ceramic membrane in this embodiment, a ceramic membrane composed of magnesium phosphate may be used as an alternative or may be formed by a freely-chosen method, such as vapor deposition or coating, in place of the anodizing process.

The above-described embodiment leads to the following inventions.

An aspect of the present invention provides an osteosynthetic implant including a base material composed of magnesium or a magnesium alloy and a ceramic membrane containing magnesium and formed on a surface of the base material. The ceramic membrane includes a porous lower membrane layer disposed in a region adjacent to the base material and an upper membrane layer covering the lower membrane layer and serving as an outermost layer. The upper membrane layer is denser than the lower membrane layer.

According to this aspect, when the osteosynthetic implant is implanted in bone tissue, the upper membrane layer serving as the outermost layer comes into contact with the bone tissue and reacts with the fluid in the body, so that decomposition commences. Since the upper membrane layer, which is relatively dense, decomposes at a low rate due to having a relatively small contact area with the fluid, the upper membrane layer remains without being decomposed and functions as a structural member that supports an affected site while bone fusion of the affected site progresses. Then, after the upper membrane layer is decomposed, the porous lower membrane layer comes into contact with the bone tissue so that the contact area with the bone tissue and the fluid increases, resulting in an increased decomposition rate. Furthermore, when the decomposition progresses and the lower membrane layer disappears, the base material subsequently comes into contact with the bone tissue so that the decomposition rate rapidly increases, whereby the base material is ultimately decomposed without remaining as a foreign object within the bone tissue.

Specifically, although the decomposition progresses relatively slowly immediately after the osteosynthetic implant is implanted into the biological organism, the decomposition rate slowly increases and the osteosynthetic implant is decomposed at the point when the mechanical strength of the structural member becomes no longer necessary as the bone fusion progresses, so that the osteosynthetic implant can ultimately decompose and disappear.

In the above aspect, the upper membrane layer preferably has a decomposition and disappearing period in which the upper membrane layer remains within a biological organism until completion of bone fusion.

Accordingly, the upper membrane layer can function as a structural member until the bone fusion is completed, and the decomposition rate can be increased to cause the upper membrane layer to quickly decompose upon completion of the bone function.

Furthermore, in the above aspect, the upper membrane layer may have a maximum pore diameter of 1 μm.

Accordingly, the body fluid within the biological organism can be prevented from reaching the base material when the osteosynthetic implant is immersed in the body fluid, so that the corrosion resistance at the initial stage of the implanting process can be improved, and the mechanical strength can be maintained while the bone fusion progresses.

Furthermore, in the above aspect, the upper membrane layer may have a thickness of 0.01 μm to 10 μm.

Accordingly, a decomposition and disappearing period of about three to twelve weeks is ensured, so that the mechanical strength can be maintained while the bone fusion progresses.

Furthermore, in the above aspect, the upper membrane layer may be amorphous, and the lower membrane layer may be a mixture of amorphous and crystalline.

Accordingly, in the amorphous upper membrane layer, intergranular corrosion does not occur, so that the corrosion resistance can be enhanced. Therefore, the decomposition rate of the upper membrane layer can be sufficiently reduced. After the upper membrane layer decomposes, intergranular corrosion of the crystalline contained in the lower membrane layer commences, so that the decomposition rate can be increased. Moreover, the fact that the lower membrane layer is mixed crystal containing an amorphous material implies that the lower membrane layer has a crystalline structure similar to that of the amorphous upper membrane layer, as compared with a case where the lower membrane layer is composed of a crystalline material alone, so that the upper membrane layer and the lower membrane layer are stably joined to each other, whereby the membrane structure of the implant material can be maintained.

Furthermore, in the above aspect, the lower membrane layer may have a pore with a maximum diameter of 1 μm or smaller.

Accordingly, the contact area between the base material and the upper membrane layer is prevented from decreasing as a result of larger pores so that the bonding strength is ensured, whereby the upper membrane layer can be stably maintained. Thus, the decomposition rate within the biological organism can be stably controlled.

Furthermore, in the above aspect, a crystal included in the lower membrane layer may have a particle diameter smaller than 500 nm.

Accordingly, the area of the crystalline interface is increased with decreasing particle diameter of the crystal so that the contact area with the body fluid is increased, whereby the decomposition rate can be increased after the upper membrane layer decomposes and disappears.

Furthermore, in the above aspect, a crystal included in the lower membrane layer may be magnesium oxide.

Accordingly, the lower membrane layer can have high biocompatibility and can also have high compatibility with the upper membrane layer and the base material, whereby the upper membrane layer and the base material can be stably joined to each other.

Furthermore, in the above aspect, the ratio of a thickness of the lower membrane layer to a thickness of the ceramic membrane may be lower than 70%.

Accordingly, the corrosion resistance can be ensured until the bone fusion is completed.

Furthermore, in the above aspect, the ceramic membrane may have a thickness of 0.1 μm to 12 μm.

Accordingly, this can minimize the overall thickness of the ceramic membrane and can prevent a chemical compound (e.g., apatite containing magnesium) produced as a result of biodegradation of the ceramic membrane and accumulating on the implant surface from inhibiting a decomposition reaction caused by contact between the body fluid and the magnesium or the magnesium alloy of the base material.

Furthermore, in the above aspect, main components of the ceramic membrane may include magnesium, phosphor, and oxygen.

Accordingly, because the ceramic membrane is composed of components contained within the biological organism, specifically, the bone serving as an affected site to be medically treated, the compatibility of the ceramic membrane with the bone can be enhanced.

### REFERENCE SIGNS LIST

1 osteosynthetic implant
2 base material
3 ceramic membrane
4 lower membrane layer
5 upper membrane layer

1. An osteosynthetic implant comprising:
a base material composed of magnesium or a magnesium alloy; and
a ceramic membrane containing magnesium and formed on a surface of the base material,
wherein the ceramic membrane includes a porous lower membrane layer disposed in a region adjacent to the base material and an upper membrane layer covering the lower membrane layer and serving as an outermost layer, the upper membrane layer being denser than the lower membrane layer.

2. The osteosynthetic implant according to claim 1,
wherein the upper membrane layer has a decomposition and disappearing period in which the upper membrane layer remains within a biological organism until completion of bone fusion.

3. The osteosynthetic implant according to claim 1,
wherein the upper membrane layer has a maximum pore diameter of 1 μm.

4. The osteosynthetic implant according to claim 1,
wherein the upper membrane layer has a thickness of 0.01 μm to 10 μm.

5. The osteosynthetic implant according to claim 1,
wherein the upper membrane layer is amorphous, and the lower membrane layer is a mixture of amorphous and crystalline.

6. The osteosynthetic implant according to claim 1,
wherein the lower membrane layer has a pore with a maximum diameter of 1 μm or smaller.

7. The osteosynthetic implant according to claim 5,
wherein a crystal included in the lower membrane layer has a particle diameter smaller than 500 nm.

8. The osteosynthetic implant according to claim 5,
wherein a crystal included in the lower membrane layer is magnesium oxide.

9. The osteosynthetic implant according to claim 1,
wherein the ratio of a thickness of the lower membrane layer to a thickness of the ceramic membrane is lower than 70%.

10. The osteosynthetic implant according to claim 1,
wherein the ceramic membrane has a thickness of 0.1 μm to 12 μm.

11. The osteosynthetic implant according to claim 1,
wherein main components of the ceramic membrane include magnesium, phosphor, and oxygen.
